(19) United States
(12) Patent Application Publication
(10) Pub. No.: US 2016/0290981 A1
(43) Pub. Date: Oct. 6, 2016

(54) Apparatus and Methods for Disintegration Testing

(71) Applicant: DISTEK, INC., North Brunswick, NJ (US)

(72) Inventors: Jeffrey Brinker, Westfield, NJ (US); Shawn Craig, Jobstown, NJ (US); Wenyu Wang, Pennginton, NJ (US)

(73) Assignee: Distek, Inc., North Brunswick, NJ (US)

(21) Appl. No.: 15/035,854

(22) PCT Filed: Nov. 12, 2014
(86) PCT No.: PCT/US2014/065331

§ 371 Date: May 11, 2016

Related U.S. Application Data
(60) Provisional application No. 61/902,536, filed on Nov. 11, 2013.

(51) Int. Cl. G01N 33/15; G01N 21/49

(52) U.S. Cl. CPC G01N 33/15; G01N 21/49

(57) ABSTRACT

In one embodiment, a disintegration or dissolution testing apparatus (100) including at least one area for a compartment (205) to hold a dosage form during disintegration testing, and at least one light emitter (302) and at least one light detector (303) arranged around each of the areas so as to face the area. The light detector (303) is adapted to provide, when a compartment (205) is disposed within the area a signal indicative of the amount of detected light provided by the light emitter (302). In another embodiment, a bottom flange (202) for disintegration test apparatus (100) is encapsulated in a material that (i) permits the bottom flange (202) to be immersed in a liquid medium during disintegration testing without the liquid contacting any of the light emitters (302) or detectors (303), and (ii) permits light from the emitter (302) to pass through the material and be received by the light detector (303). The apparatus (100) comprises as well a drive assembly for driving the compartment (205) between a lowered position immersing the dosage form in the liquid medium and a raised postion.

### FIELD OF THE INVENTION

The present invention relates generally to scientific testing and, in particular, to disintegration testing to determine the time that tablets or capsules placed in a liquid medium take to disintegrate.

### BACKGROUND OF THE INVENTION

Taking tablets or capsules orally remains one of the most effective means of drug delivery available. The effectiveness of such dosage forms relies on the drug's absorption into fluids of the body. However, in order for the active drug in a tablet or capsule to become available for absorption into the body, a tablet or capsule must first disintegrate into smaller particles so that the drug can be discharged into bodily fluids. The process of disintegration is also important because it yields an increased surface area for the activities of certain drug particles that act locally within the gastrointestinal tract, such as tablets or capsules that contain antacids and antidiarrheals.

To ensure that disintegration is properly occurring in vivo, disintegration testing is performed in vitro using a reproducible and standardized method, namely, by confirming that tablets or capsules disintegrate within a prescribed time when placed in a liquid medium, using disintegration testing apparatus. The dosage form is placed inside a basket assembly or other United States Pharmacopeia (USP)-defined apparatus, which is lowered by the disintegration testing apparatus into a solution that emulates conditions inside the human body. The basket assembly is then raised and lowered into the solution by the disintegration testing apparatus, e.g., at a commonly-used rate of 30 times (dips) per minute.

Conventionally, a user was required to visually confirm that a tablet or capsule fully disintegrated after a certain amount of time. However, this manual intervention introduced an element of human error into the testing process.

Several automated methods now exist for determining when the dosage form is disintegrated, referred to as “auto-detection.”

In one such method, a closed circuit design is employed, whereby a metal ring is placed in a commonly-used USP fluted disk. The fluted disk is used in disintegration to hold down dosage forms that may have a tendency to float. On the bottom of the disintegration basket, a metal mesh screen is disposed to hold the dosage form and prevent it from falling out. For this method of auto-detection, the screen on the bottom of the basket is divided in two separate portions separated by a gap. Once the fluted disk (which stays on top of the dosage form) with the metal ring contacts the split screen on the bottom of the basket, a circuit is closed, and the dosage form is determined to have disintegrated.

Another such method employs the Hall effect principle, i.e., the production of a voltage difference across an electrical conductor, transverse to an electric current in the conductor and a magnetic field perpendicular to the current. In this method, a magnetic field is generated, and a Hall-effect sensor is used to determine the proximity of the fluted disk to the bottom of the basket. As the fluted disk gets nearer and nearer to the bottom of the basket, it is interpreted that the dosage form is getting smaller and smaller. Once the fluted disk is determined to have reached the bottom of the basket by Hall-effect detection, the dosage form is determined to have disintegrated.

The foregoing auto-detection methods involve mechanical components that introduce the potential for error, wear, and failure, in addition to having other disadvantages.

### SUMMARY

Embodiments of the present invention provide apparatus and methods for the disintegration testing of tablets or capsules in a liquid medium. Some embodiments employ an auto-detection method for determining the disintegration of dosage forms by using infrared emitters and detectors. Some embodiments include a bottom flange (a structural support for the bottom of basket assembly) that is fabricated to have a printed circuit board (PCB) entirely encapsulated within the bottom flange, to protect electronic components from the liquid medium. Some embodiments include a direct-current (DC) motor that rotates in one direction to effect upward motion of the basket assembly, and in the opposite direction to effect downward motion of the basket assembly, where a lead screw and nut is used to translate the motor's rotational motion to a linear motion.

In a first embodiment, the present invention provides a disintegration testing apparatus including at least one area for a compartment, and at least one light emitter and at least one light detector arranged around each of the areas so as to face the area. The compartment is adapted to hold a dosage form during disintegration testing. The at least one light detector is adapted to provide, when a compartment is disposed within the area, a signal indicative of the amount of detected light provided by the at least one light emitter.

In a second embodiment, the present invention provides a disintegration testing apparatus including a base unit having a plurality of modules used for disintegration testing. Each module includes a vessel for holding a liquid medium, a support driven by a drive assembly and adapted to travel along a vertical path, and a basket assembly detachably mountable to the support and having at least one compartment adapted to hold a dosage form during disintegration testing. The drive assembly is adapted to cause the basket assembly to travel between a lowered position immersing the dosage form in the liquid medium of the vessel and a raised position.

In a third embodiment, the present invention provides a disintegration testing apparatus including a vessel for holding a liquid medium, a support driven by a drive assembly to travel along a vertical path, and a basket assembly detachably mountable to the support and having at least one compartment adapted to hold a dosage form during disintegration testing. The drive assembly includes a lead screw and a screw nut coupled to the support and having an inside thread that matches an outside thread of the lead screw. Rotation of the lead screw in one rotational direction causes linear movement of the screw nut in a first direction, thereby causing the basket assembly to travel toward a lowered position immersing the dosage form in the liquid medium of the vessel. Rotation of the lead screw in the other rotational direction causes linear movement of the screw nut in a second direction opposite from the first direction, thereby causing the basket assembly to travel toward a raised position where the dosage form is not immersed in the liquid medium of the vessel.

In a fourth embodiment, the present invention provides a bottom flange for disintegration testing equipment. The bottom flange includes a substrate having at least one area for a respective compartment, at least one light emitter and at least one light detector arranged around each area and mounted to the substrate, and a material encapsulating the at least one light emitter, the at least one light detector, and the substrate to permit the bottom flange to be immersed in a liquid medium during disintegration testing without the liquid contacting any of the at least one light emitter and at least one light detector. The compartment is adapted to hold a dosage form during disintegration testing.

In a fifth embodiment, the present invention provides an auto-detection apparatus for disintegration, including at least one light emitter and at least one light detector arranged around and facing an area for a compartment. The compartment is adapted to hold a dosage form during disintegration testing. The at least one light detector is adapted to provide, when a compartment is disposed within the area, a signal indicative of the amount of detected light provided by the at least one light emitter.

In a sixth embodiment, the present invention provides a method for manufacturing a bottom flange for disintegration testing equipment. The method includes: arranging at least one light emitter and at least one light detector around an area of a substrate so as to face the area; mounting the at least one light emitter and the at least one light detector to the substrate; and encapsulating the at least one light emitter, the at least one light detector, and the substrate in a material that permits the bottom flange to be immersed in a liquid medium during disintegration testing without the liquid contacting any of the at least one light emitter and at least one light detector.

In a seventh embodiment, the present invention provides a method for auto-detection in disintegration testing. The method includes: providing a signal from at least one light detector, the signal indicative of the amount of detected light provided by at least one light emitter. The at least one light emitter and at least one light detector are arranged around and facing an area for a compartment. The compartment is adapted to hold a dosage form during disintegration testing.

### BRIEF DESCRIPTION OF THE DRAWINGS

FIG. 1 shows a front perspective view of a disintegration testing apparatus, in one embodiment of the invention;

FIG. 2 shows a front perspective view of a basket assembly of the disintegration testing apparatus of FIG. 1;

FIG. 3 shows a front perspective view of the bottom flange of the basket assembly of FIG. 2;

FIG. 4 shows a plan view of the component layout of the PCB board of FIG. 2;

FIG. 5 shows a top perspective view of a portion of the encapsulated bottom flange of the basket assembly of FIG. 2;

FIG. 6 shows a portion of one of the modules of the disintegration testing apparatus of FIG. 1;

FIG. 7 is an enlarged view of a portion of the module of FIG. 6;

FIG. 8 is a flowchart of a first exemplary method implemented by the disintegration testing apparatus of FIG. 1 for calibration of the disintegration testing apparatus and performance of a disintegration test;

FIG. 9 is a flowchart of a second exemplary method implemented by the disintegration testing apparatus of FIG. 1 for calibration of the disintegration testing apparatus;

FIG. 10 is a flowchart of a second exemplary method implemented by the disintegration testing apparatus of FIG. 1 for performance of a disintegration test;

FIG. 11 is a flowchart of a third exemplary method implemented by the disintegration testing apparatus of FIG. 1 for performance of a disintegration test;

FIG. 12 is a flowchart of an exemplary initialization operation of the disintegration-testing apparatus;

FIG. 13 is a flowchart of an exemplary method-run operation of the disintegration-testing apparatus;

FIG. 14 shows an exemplary general settings window on the touch screen;

FIG. 15 shows an exemplary new method creation window on the touch screen; and

FIG. 16 shows an exemplary operations window on the touch screen.

### DETAILED DESCRIPTION

Described below is an embodiment of the invention, implemented as a centrally controlled modular disintegration-testing apparatus having a base unit with a controller and one or more disintegration-testing modules, each module employing one basket assembly that has six compartments in the form of glass tubes that are repeatedly lowered into a beaker (or other vessel) containing a test medium. Using a scalable, centrally controlled modular disintegration-testing apparatus can provide various advantages over the conventional unitary integrated apparatus. Users who require a disintegration testing system having fewer than six modules will be able to have such a system and possibly incur reduced costs. If the user's needs grow, additional disintegration-testing modules may be added as needed. If a single disintegration-testing module of the modular system breaks down, then replacement of the single module is simpler, faster, and cheaper than the repair or replacement of an entire unitary multi-beaker system or bath-based system. Furthermore, additional novel features of a central controller, which are described below, add convenience and utility to the modular disintegration-testing apparatus.

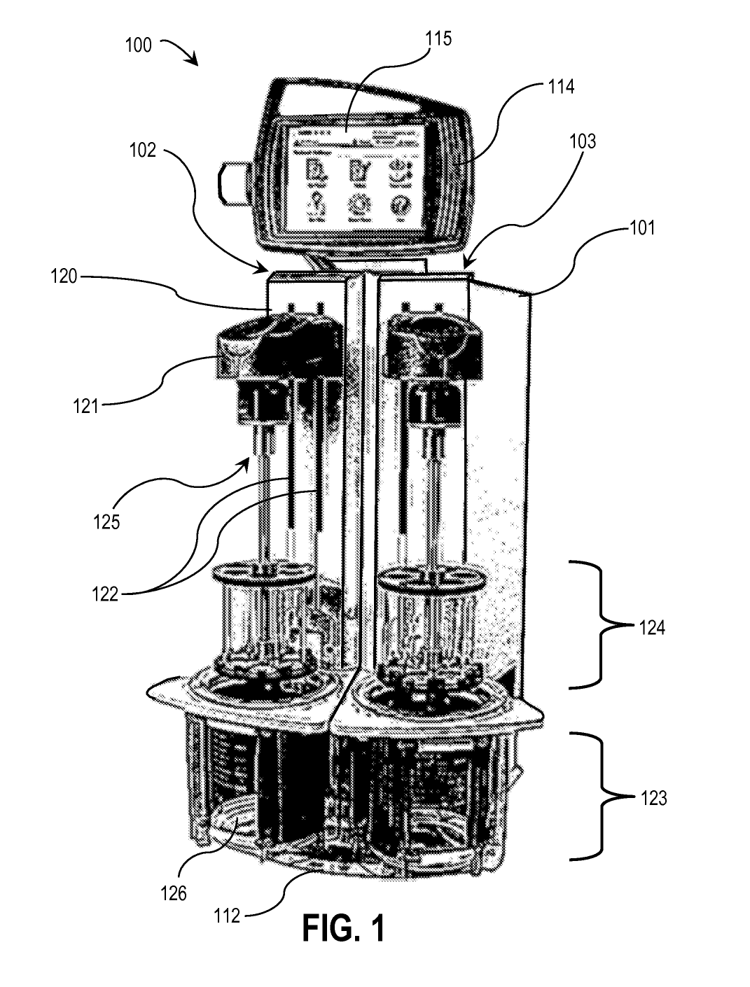

FIG. 1 shows a front perspective view of modular disintegration-testing apparatus 100 in accordance with one embodiment of the present invention. Apparatus 100 is scalable for use with either one or two detachable disintegration-testing modules. Apparatus 100 includes base unit 101 and detachable disintegration-testing modules 102 and 103.

Base unit 101 is shaped substantially as a vertical tower or stand with a supportive base plate 112. Base unit 101 includes two attachment bays (not shown) along part of its periphery, which are receptacles adapted to hold in place corresponding detachable disintegration-testing modules (such as, for example, modules 102 and 103) or unused-attachment-bay covers (not shown). Each attachment bay includes a connection port (not shown) for interfacing with an attached disintegration-testing module. (Although this embodiment includes only two attachment bays, in alternative embodiments, base unit 101 can accommodate up to four additional disintegration-testing modules in addition to modules 102 and 103, for a total of six modules.) To connect an additional disintegration-testing module, the user removes an unused-attachment-bay cover (not shown) and inserts and connects the module into the corresponding attachment bay. Disintegration-testing modules 102 and 103 are secured to their respective attachment bays with screws.

Base unit 101 also includes a controller (not shown) that has a processor and a communicatively connected memory usable to store program code, parameters, measurements, and other useful information. Base unit 101 additionally includes circuitry to communicatively connect the controller to various elements of apparatus 100, such as, for example, the connection ports of the attachment bays and/or a serial communication port (e.g., RS-232 or USB) for interfacing with an external computer.

A user interface 114 is disposed at the top of base unit 101. User interface 114 includes a touch screen 115 communicatively connected to the controller. User interface 114 may include a user-interface controller (not shown) communicatively connected to the controller of base unit 101 and to touch screen 115. The modules may be labeled with unique and visible identifiers, such as sequential integer numbers (e.g., 1 and 2), to correlate respective disintegration-testing modules and/or components therein with their corresponding iconic representations on touch screen 115.

The description below provided for disintegration-testing module 102 also applies to other disintegration-testing modules, such as module 103.

Disintegration-testing module 102 connects electronically to base unit 101 via a connection port in the corresponding attachment bay of base unit 101 for the receipt of electrical power from base unit 101 and for communication between module 102 and the controller of base unit 101. The connection port may be in the form of a standard computer peripheral component interconnect (PCI) card, where the corresponding connection port of the attachment bay is a corresponding PCI slot.

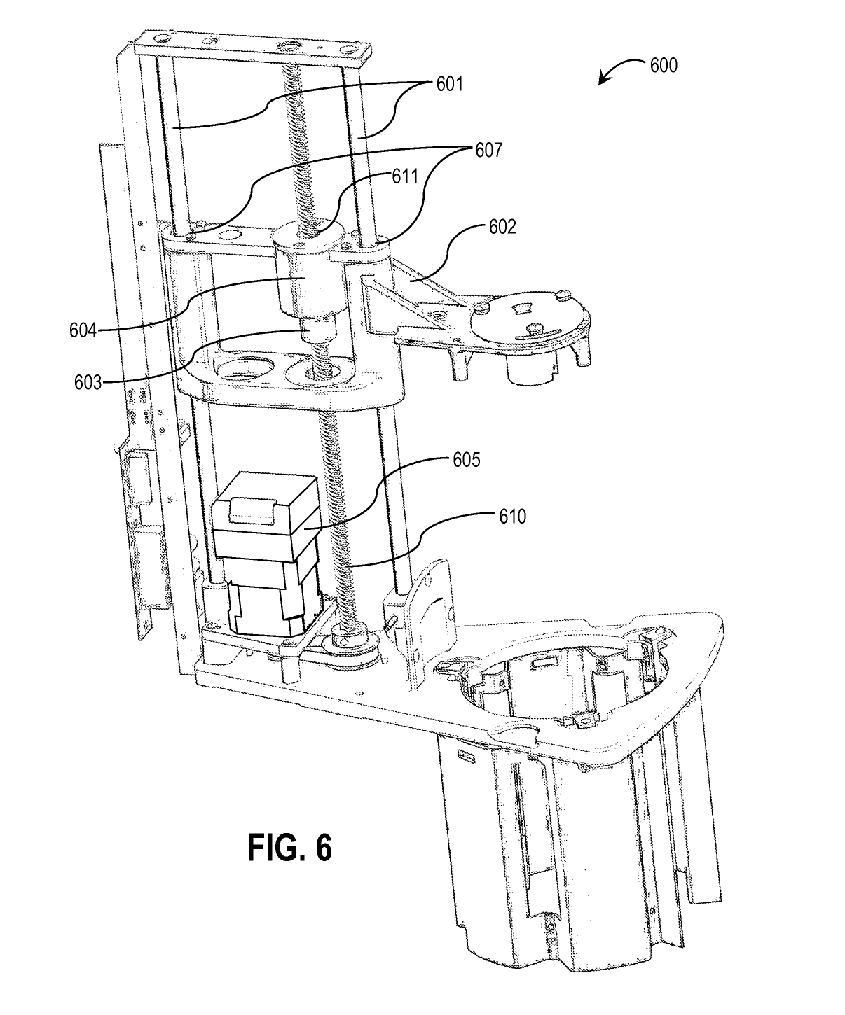

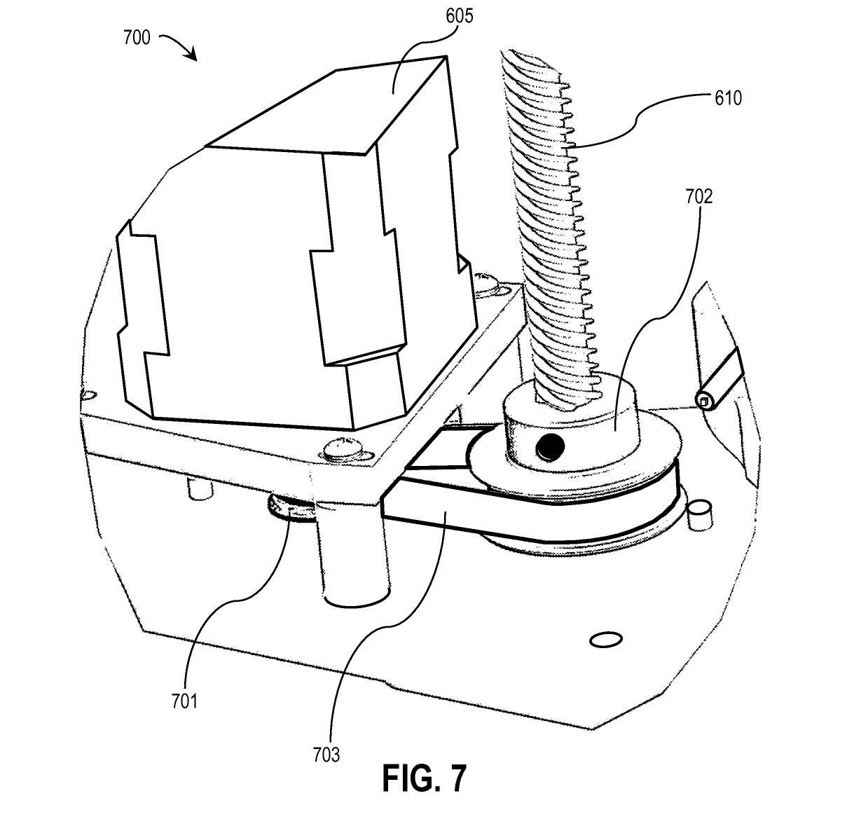

Disintegration-testing module 102 includes a housing 120 and a basket assembly support 121 that travels vertically relative to housing 120. Support 121 holds a basket assembly 125, which detachably connects to the underside of support 121 via mechanical and electrical couplings. Support 121 is supported by a drive assembly (shown in FIGS. 6-7) located within housing 120. Based on control signals from the controller of base unit 101, the drive assembly moves basket assembly 125 vertically, along a pair of parallel tracks 122 formed in housing 120, between a lowered position 123 inside beaker 126 and a raised position 124. Beaker 126 has a heating jacket (not shown) wrapped around at least a portion of beaker 126. The heating jacket includes heating elements for providing heat to beaker 126 and its contents. Any type of heating elements (e.g., resistance wires) may be used by the heating jacket. Beaker 126 also has a temperature sensor (not shown), which is used by the controller of base unit 101 in a negative feedback loop to allow for the setting of one or more precise operating temperatures for the contents of beaker 126, where the operating temperature may be rapidly reached and then stabilized. The operating temperature is controlled by the controller of base unit 101, which also receives, as input, the temperature sensor data. A typical operating temperature for the contents of beaker 126 is 37 degrees Celsius. Use of separate beakers 126, each with its own temperature control, eliminates conventional problems associated with standard water baths, such as slow heating rates and algae growth. The use of an integrated temperature sensor enables the monitoring and documenting of the individual breaker temperatures for improved regulatory compliance while eliminating the need for manually verifying temperature equilibration prior to dosage form introduction.

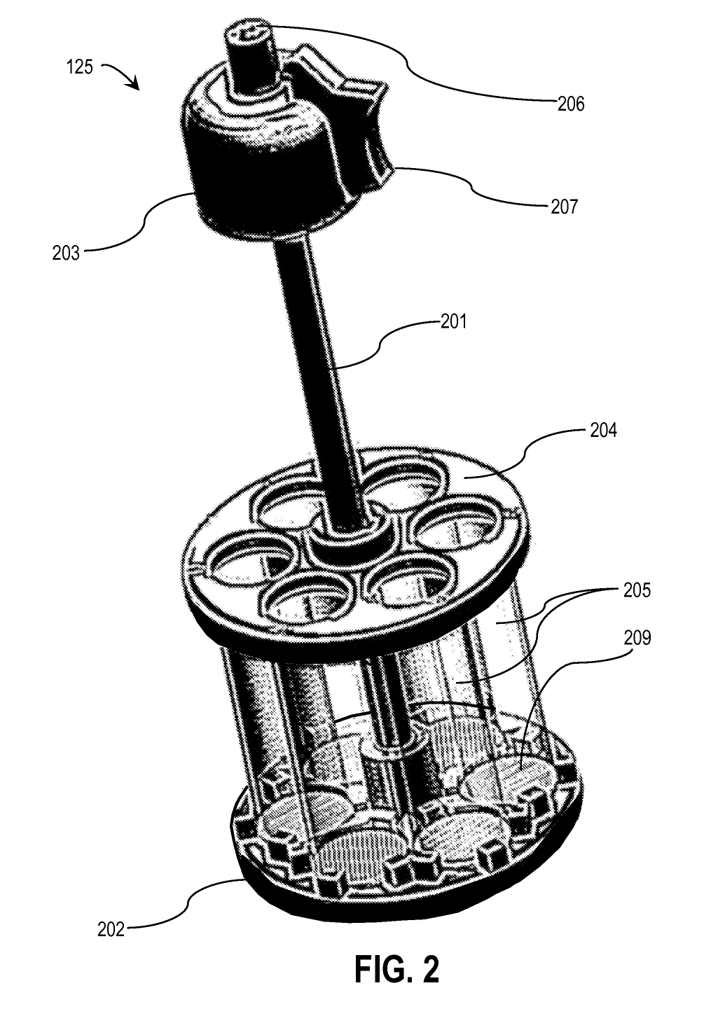

FIG. 2 shows basket assembly 125, in one embodiment of the invention. Basket assembly 125 includes a shaft 201 having a bottom flange 202 at one end, a mount 203 at the other end, and a top flange 204 disposed along shaft 201. Top flange 204 is disposed along shaft 201 at a height that permits top flange 204 to support six glass tubes 205 inside which dosage forms are disintegrated in a liquid medium during testing. Each tube has a screen 209 disposed at its base. The dosage form being tested rests on screen 209 while basket assembly 125 is raised. During the testing process, the dosage form floats upward as basket assembly 125 is lowered into the liquid medium. The dosage form floats downward to rest on screen 209 as basket assembly 125 is raised from the liquid medium. Bottom flange 202 supports the bases of glass tubes 205. Mount 203 includes an electrical connector 206 (e.g., 3-wire) that permits signals to be exchanged between basket assembly 125 and the controller, to provide sensing and control functionality during testing. Mount 203 also includes a mechanical connector, such as spring-biased rocker 207, which latches basket assembly 125 securely into a corresponding mechanical slot (not shown) on the underside of support 121, so as to eliminate undesirable sway during testing.

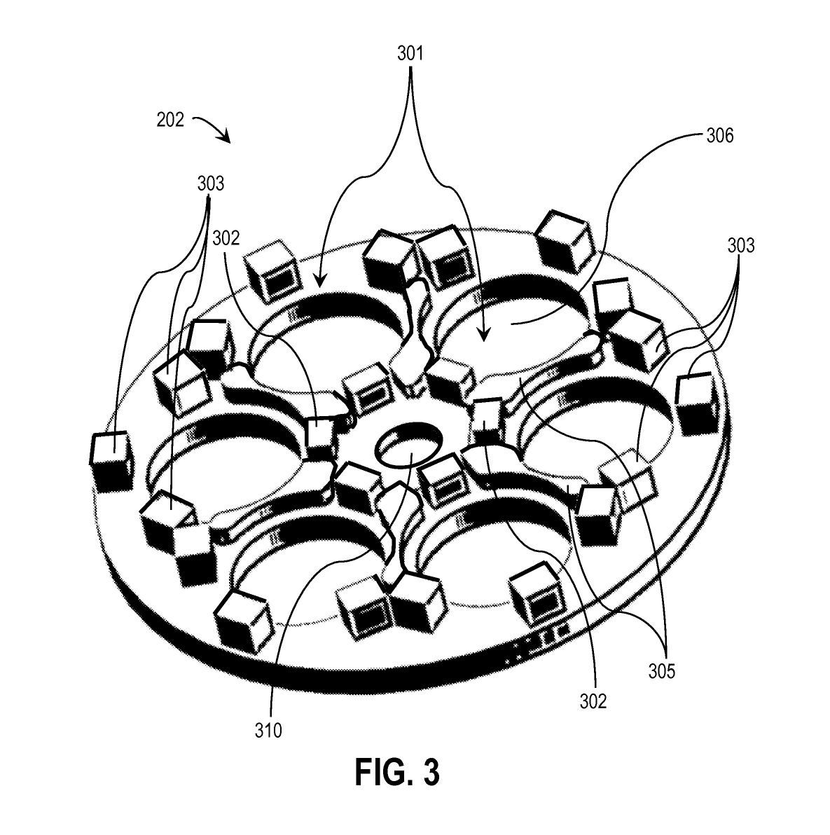

FIG. 3 shows bottom flange 202, which includes six receptacles 301, each for holding in place the base of a respective glass tube 205, and a central hole 310 for attachment of bottom flange 202 to shaft 201. Each receptacle 301 is formed from a pair of side supports 305 disposed above an aperture 306 formed in bottom flange 202 that allows ambient light to pass through bottom flange 202 and to reach glass tube 205. (In alternative embodiments, the receptacle can simply be an area where the glass tube 205 rests or is otherwise disposed and does not necessarily include physical features for retaining the glass tube 205, such as side supports 305.) As shown, a respective invisible light emitter 302 and three respective invisible light detectors 303 are arranged around each receptacle 301 so as to face the base of glass tube 205, which houses the dosage form. In some alternative embodiment, instead of employing six smaller glass tubes, a bolus basket is used, which employs three larger glass tubes (e.g., double the diameter of glass tubes 205) that are evenly spaced and supported by top and bottom flanges. In such embodiments, due to the increased surface area of each glass tube, larger quantities (e.g., double the quantities) of invisible light detectors and and/or invisible light emitters are desirably used, e.g., six invisible light detectors and two invisible light emitters (or one brighter invisible light emitter).

Emitters 302 and detectors 303 may include, e.g., an infrared emitter and detector, such as the TSKS5400S infrared 950 nm emitting diode and the BPW41N high-speed, high radiant-sensitivity PIN photodiode manufactured by Vishay Semiconductors, or part no. QSE773, which is a 920NM IC PIN photodiode manufactured by Fairchild Semiconductor. In alternative embodiments, emitters 302 and detectors 303 may also include an LED and phototransistor; a near-infrared emitter and detector, such as a laser-diode source and a laser diode-based sensor; an ultraviolet source and sensor; or another type of invisible-light or visible-light source and sensor. Emitters 302 are all centrally disposed and face outwardly, away from central hole 310. Detectors 303 are all disposed near the periphery of bottom flange 202 and face inwardly, towards central hole 310.

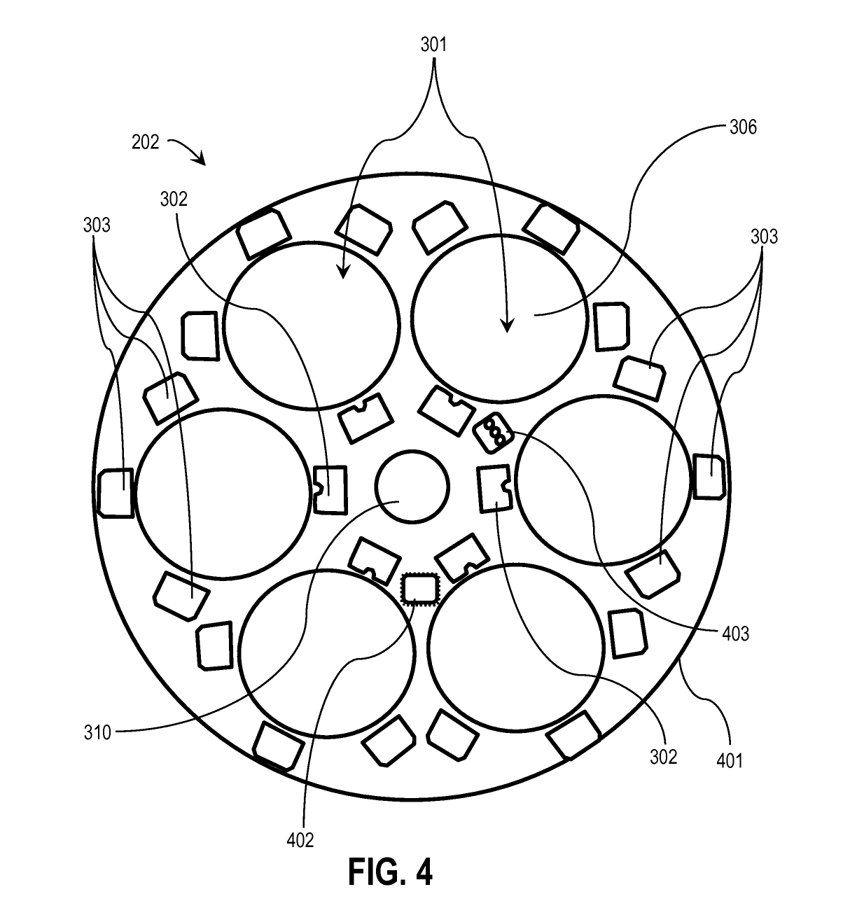

FIG. 4 shows the PCB layout for bottom flange 202, where emitters 302 and detectors 303 are mounted to a PCB 401 (or other substrate). PCB 401 also includes a microcontroller 402 coupled to control the operations of emitters 302 and detectors 303 and in communication with the controller of base unit 101. Microcontroller 402 acquires digitalized data indicative of changes in light within each of glass tubes 205 and sends the data to the controller of base unit 101. The data may be sent, e.g., over a serial communications link via electrical connector 403 (e.g., 3-wire), which serves as a connection for a wiring harness or the like, permitting communications between the circuit of PCB 401 and the controller of base unit 101.

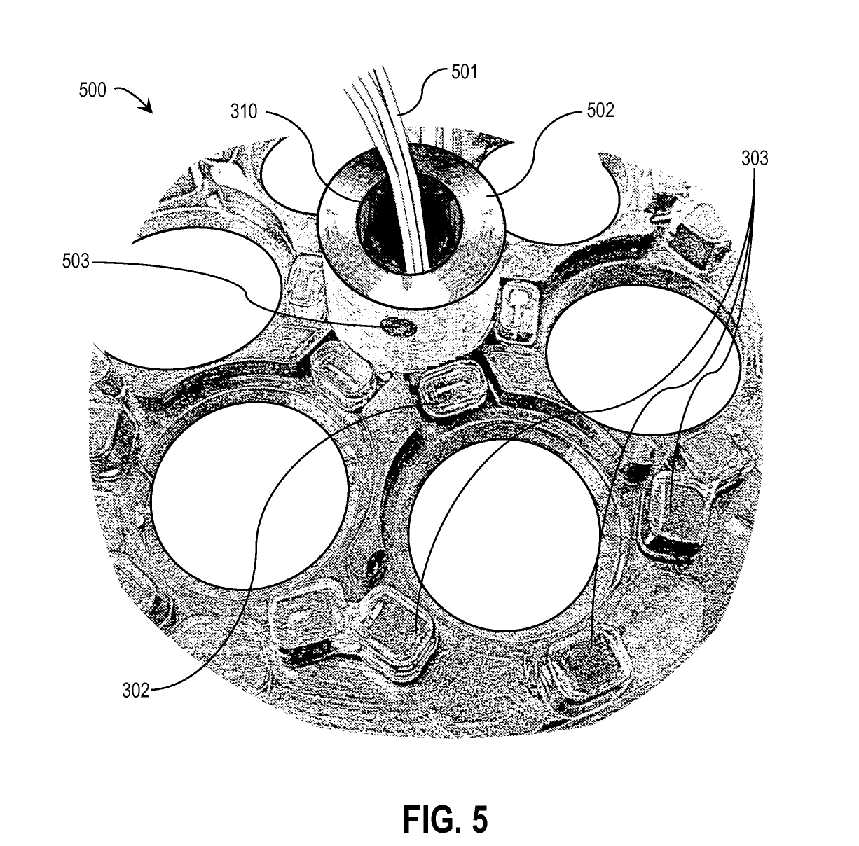

Bottom flange 202 is completely submerged in a liquid medium during testing. FIG. 5 shows a portion 500 of bottom flange 202 after a step during the manufacturing process in which PCB 401 and all of the components mounted on PCB 401 are entirely encapsulated within bottom flange 202, and sleeve 502 is fastened (e.g., bolted through central hole 310) to PCB 401. Set screw threads 503 are formed in sleeve 502 to receive a screw for securing bottom flange 202 to shaft 201.

Encapsulation protects PCB 401 and its various components from the liquid medium, while allowing bottom flange 202 to serve as a structural support for the bottom of basket assembly 125. Encapsulation is performed during the assembly process, wherein PCB 401 is placed in a mold and dyed, and plastic urethane is injected. (Other materials for encapsulation may be used in alternative embodiments.) The dye allows invisible light to pass through and not interfere with the operation of emitters 302 and detectors 303. Wiring 501 from electrical connector 403 passes through central hole 310. All gaps around wiring 501, around set screw threads 503, and inside central hole 310 are filled for a complete seal. After completion of the encapsulation process, the encapsulated bottom flange 202 meets USP guidelines for the bottom flange of a disintegration basket.

To simulate the dynamics that a dosage form experiences in the human anatomy, basket assembly 125 is typically raised and lowered at 30 strokes (dips) per minute, into and out of a solution in beaker 126. In one embodiment, this is achieved using an arrangement as shown in FIGS. 6 and 7.

FIG. 6 shows a portion 600 of module 102 disintegration-testing apparatus 100, in one embodiment of the invention. Portion 600 includes a drive assembly that moves basket assembly 125 vertically, along tracks 122, between lowered position 123 inside beaker 126 and raised position 124. The drive assembly includes a pair of vertical guide rods 601 on which a spindle plate 602 is slidably disposed via cylindrical apertures 607 formed in spindle plate 602. The drive assembly further includes a brushless DC motor 605, a lead screw 610, and a lead screw nut 603 and nut adapter 604 disposed thereon. Nut adapter 604, which is slidably disposed on lead screw 610 via a cylindrical aperture 611, couples lead screw nut 603 to spindle plate 602. The inside diameter of cylindrical aperture 611 is greater than the outside diameter of lead screw 610, and, as a result, nut adapter 604 does not come into direct contact with lead screw 610. Lead screw nut 603, which has an internal cylindrical aperture (not shown) that is threaded to complement and correspond to the threads on the outer surface of lead screw 610, does come into direct contact with lead screw 610. Lead screw 610 is adapted to be rotationally powered by motor 605. Guide rods 601 are substantially parallel to lead screw 610. Disintegration-testing module 100 may include additional guide rods. The guide rods are used to provide stability to disintegration-testing module 102 and guide the motion of basket assembly 125. The foregoing arrangement permits lead screw nut 603 to drive spindle plate 602 upwards or downwards along lead screw 610, based on the rotation of lead screw 610. The controller of base unit 101 may control the speed (i.e., RPM) of the motor, thereby controlling the speed at which spindle plate 602, and hence basket assembly 125, moves up or down lead screw 610.

FIG. 7 shows additional details via an enlarged view 700 of portion 600. As shown, motor 605 rotates in one direction for upward motion of basket assembly 125, and in the opposite direction for downward motion of basket assembly 125. Accordingly, motor 605 repeatedly reverses direction during operation. Motor 605 has a motor pulley 701 coupled to the shaft of motor 605, and lead screw 610 has a lead screw pulley 702 disposed at its base. A belt 703 connects motor pulley 701 to lead screw pulley 702 so that, when motor pulley 701 rotates, lead screw 610 is caused to rotate. Lead screw nut 603 and nut adapter 604 translate this rotational motion to a linear motion that raises or lowers spindle plate 602, depending on the direction of rotation of the spindle (not shown) of motor 605.

Two different modules 102, 103 are employed in this embodiment of disintegration-testing apparatus 100, even though portion 600 corresponds to only one of those modules and controls only one basket assembly 125. In order to facilitate the control of two different basket assemblies in a single disintegration-testing apparatus, modules 102 and 103 are independent of each other, but work in harmony. Each basket assembly 125 has a separate module 102, 103 respectively associated with it, and therefore, each basket assembly 125 has a separate motor 605, thereby permitting different testing criteria to be used for different modules.

The controller of base unit 101 (or, in alternative embodiments, microcontroller 120) employs one or more algorithms to analyze changes in light, to determine whether the dosage form in each of glass tubes 205 (or, in some embodiments, fewer than all of glass tubes 205) has disintegrated.

In one embodiment, a method for determining whether a dosage form has disintegrated comprises using the data received from microcontroller 120 to:

(A) detect whether the dosage form is moving; and

(B) detect whether the dosage form is blocking the light.

If the data from the three respective invisible light detectors 303 shows that the dosage form (A) is no longer moving, based on a lack of fluctuations of light sensed from a respective invisible light emitter 302 at by all three of the corresponding three respective invisible light detectors 303, and (B) is no longer blocking the light from a respective invisible light emitter 302 at any of the corresponding three respective invisible light detectors 303, then a determination is made that the dosage form has disintegrated. In other embodiments, only criterion (A), only criterion (B), or alternative or additional criteria, are used to determine whether the dosage form has disintegrated.

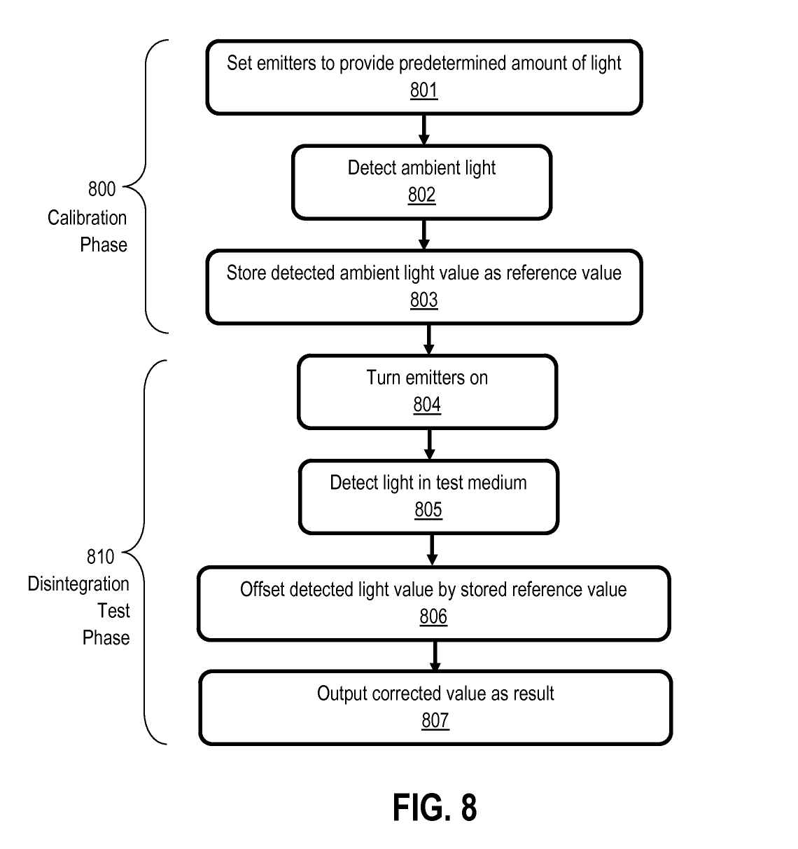

FIG. 8 is a flowchart of a first exemplary method implemented by disintegration testing apparatus 100, in one embodiment of the invention, including a calibration phase 800 and a disintegration testing phase 810. In the exemplary method of FIG. 8, prior to the initiation of the auto-detection process, calibration routine phase 800 is automatically performed to subtract ambient background light and reduce the potential influence of outside lighting on the detection process. In this scenario, before the detection process begins, emitters 302 are set to provide a predetermined amount of light (e.g., no light, little light, or maximum light, in different embodiments) as a baseline (step 801), and the amount of ambient light is detected (step 802) by invisible light detectors 303 and is stored (step 803) as a reference value to be used during the normal detection process. This ends calibration phase 800. During disintegration test phase 810, emitters 302 are turned back on (step 804), and the light in the test medium is then detected (step 805). The detected light in the test medium is offset (step 806) by the stored reference value obtained during calibration phase 800, and the corrected value is output as a result (step 807). Steps 804-807 are repeated during the entire disintegration test phase 810, such that the reference value representing the amount of ambient light detected during calibration phase 800 is subtracted from subsequent readings taken by invisible light detectors 303. In some embodiments, only a disintegration testing phase is employed, without any calibration phase. Different embodiments may involve performing calibration and testing at different times and intervals.

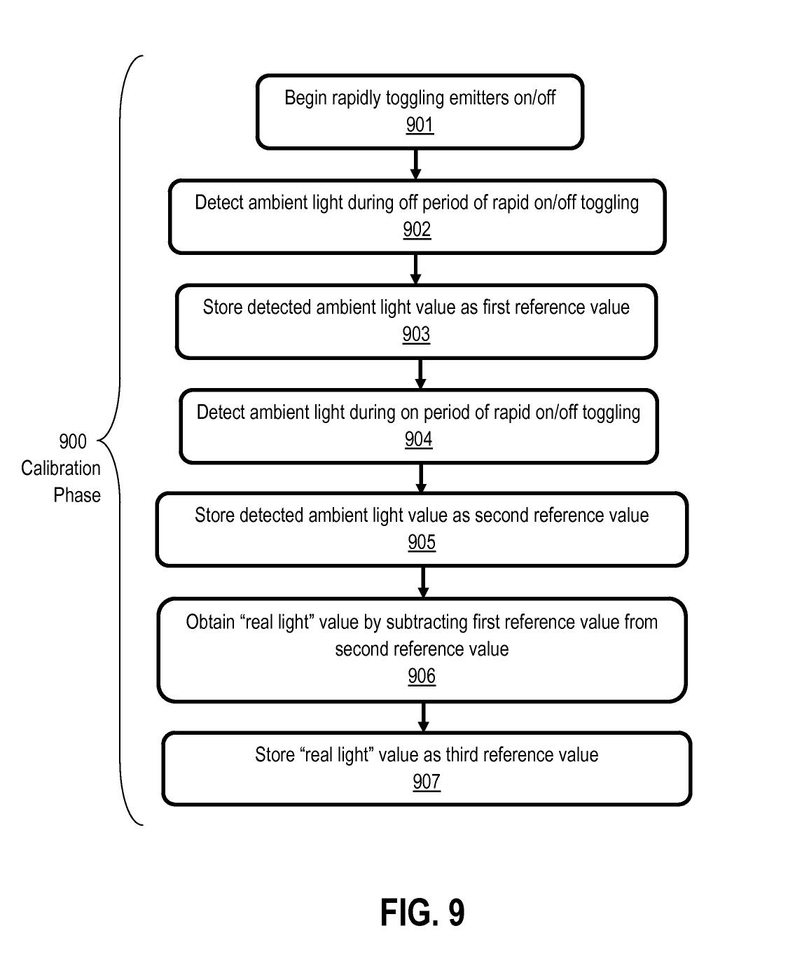

In some embodiments, as illustrated in FIGS. 9-11, “rapid toggling” type calibration and detection may additionally or alternatively be performed. In this scenario, emitters 302 are rapidly toggled alternatively between an on and off state based on a predetermined timing pattern. (For example, the “on” and “off” states each have the same fixed duration between 2 and 300 ms, in one embodiment; or the “on” and “off” states have different fixed durations between 2 and 300 ms, in another embodiment). This rapid toggling type calibration may take place during either the calibration routine, the detection process, or both. (Although the term “rapid” is used to refer to such a predetermined timing pattern, such a timing pattern could alternatively employ relatively long durations for the “on” and “off” states, in alternative embodiments.)

FIG. 9 is a flowchart of a second exemplary method implemented by disintegration testing apparatus 100 for calibration of disintegration testing apparatus 100, in one embodiment of the invention, consisting of calibration phase 900. In the exemplary method of FIG. 9, prior to the initiation of the auto-detection process, calibration routine phase 900 is automatically performed to subtract ambient background light and reduce the potential influence of outside lighting on the detection process. First, rapid on/off toggling of emitters 302 begins (step 901). During an “off” period of the on/off toggling, the amount of ambient light is detected (step 902) by invisible light detectors 303 and stored (step 903) as a first reference value. Next, during an “on” period of the on/off toggling, the amount of ambient light is detected (step 904) by invisible light detectors 303 and stored (step 905) as a second reference value. Next, the total amount of ambient light that was detected while emitters 302 were off is subtracted (step 906) from the total amount of ambient light detected while emitters 302 were turned back on, resulting in a “real light” value that can be used during the detection algorithm, which is stored (step 907) as a third reference value. This “real light” value represents the actual amount of light being provided by the emitters 302 being toggled and is used by the detection algorithm to determine, for example, when an emitter 302 is being blocked by a dosage form. The more frequently rapid toggling type calibration is performed, the more potential influences of outside dynamic lighting on the detection process can be reduced.

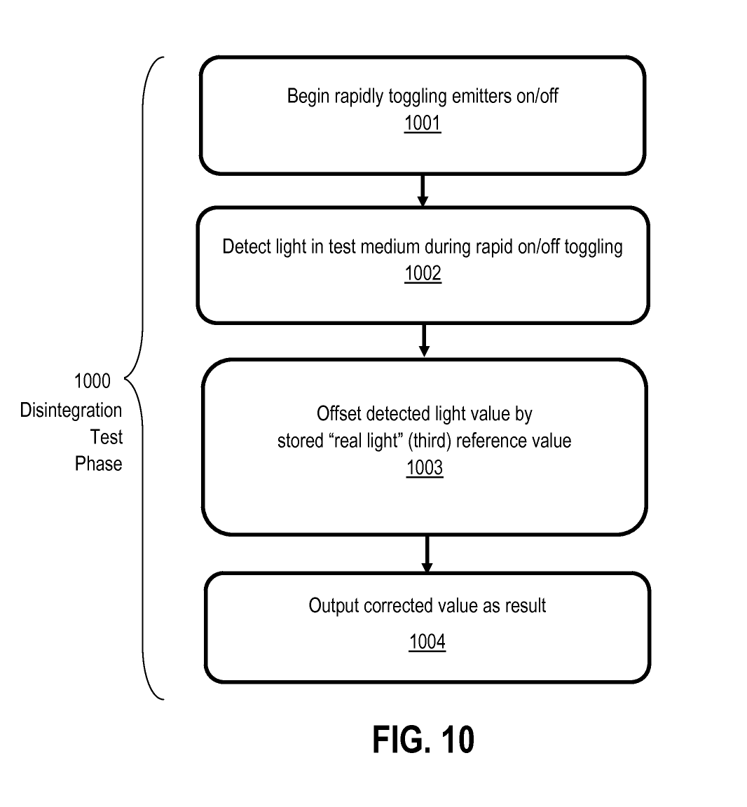

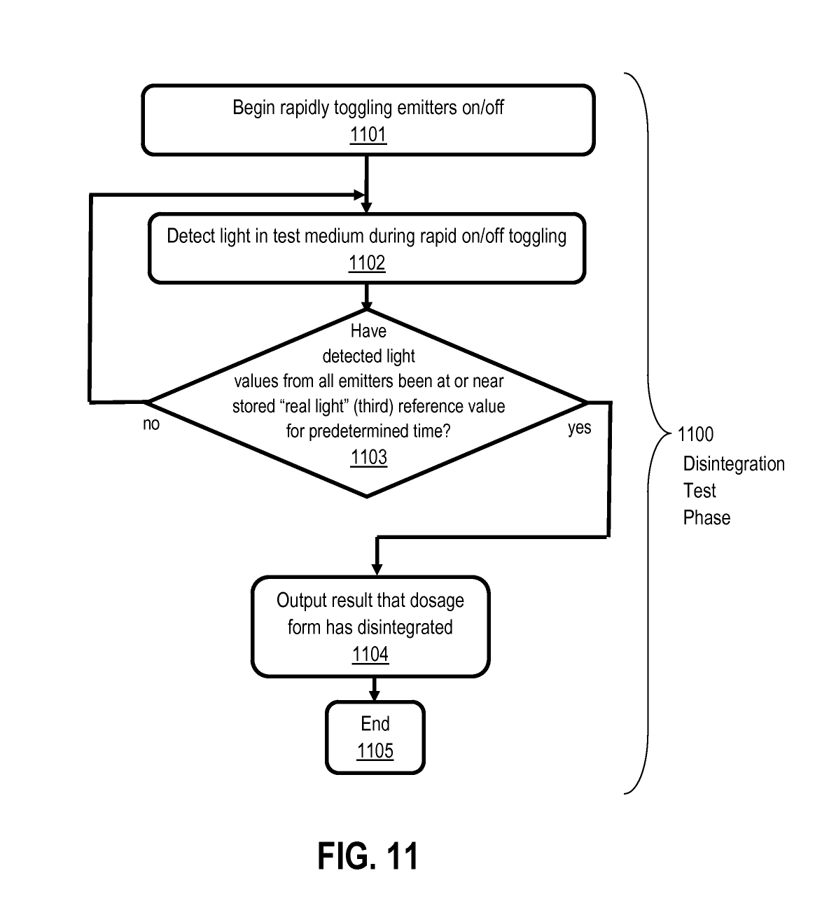

After the second exemplary method of FIG. 9 has been performed by disintegration testing apparatus 100, one or more methods for disintegration testing, such as the methods of either FIG. 10 or FIG. 11, is performed.

FIG. 10 is a flowchart of a second exemplary method implemented by disintegration testing apparatus 100 for performance of a disintegration test, in one embodiment of the invention, consisting of disintegration test phase 1000. In the exemplary method of FIG. 10, prior to the initiation of disintegration test phase 1000, calibration routine phase 900 (shown in FIG. 9) has already been performed to obtain and store a “real light” value that disintegration test phase 1000 uses to subtract ambient background light and reduce the potential influence of outside lighting on the detection process. First, rapid on/off toggling of the emitters 302 begins (step 1001). During the on/off toggling, the amount of light in the test medium is detected (step 1002) by invisible light detectors 303. Next, the detected light value is offset (step 1003) by the stored “real light” (third reference) value. The corrected value is output as a result (step 1004). Steps 1001-1004 are repeated during the entire disintegration test phase 1000, such that the “real light” value representing the amount of ambient light detected during calibration phase 900 is subtracted from subsequent readings taken by invisible light detectors 303. In this method, the corrected value output in step 1004 is used by the controller for further processing, e.g., to determine whether dosage form (A) is no longer moving and (B) is no longer blocking the light from a respective invisible light emitter 302 at any of the corresponding three respective invisible light detectors 303.

Other methods may be used to detect disintegration of a dosage form. For example, FIG. 11 is a flowchart of a third exemplary method implemented by disintegration testing apparatus 100 for performance of a disintegration test, in another embodiment of the invention, consisting of disintegration test phase 1100. In the exemplary method of FIG. 11, prior to the initiation of disintegration test phase 1100, calibration routine phase 900 (shown in FIG. 9) has already been performed to obtain and store a “real light” value that is used during disintegration test phase 1100. First, rapid on/off toggling of emitters 302 begins (step 1101). During the on/off toggling, the amount of light in the test medium is detected (step 1102) by invisible light detectors 303. Next, a determination is made (step 1003) whether the detected light values from all emitters 302 have been at or near the stored “real light” (third reference) value for a predetermined period of time. If not, then the method returns to step 1102 to obtain a new value for the amount of light in the test medium. If so, then the method proceeds to step 1104. A result that the dosage form has disintegrated is provided (step 1104), and the method ends (step 1105). In this method, instead of outputting a corrected value to be processed by the controller, a simple binary value is output to indicate whether or not the dosage form has disintegrated.

The controller of base unit 101 of FIG. 1 can control any set of connected disintegration-testing modules. In other words, the controller may specify operation of a single disintegration-testing module or of one or more different subsets of a plurality of disintegration-testing modules. When controlling multiple modules, the controller may independently control each of the modules. In other words, the controller may conduct a first operation on one of disintegration-testing modules 102, 103 at a first rate (i.e., dips per minute), and a second operation on the other of disintegration-testing modules 102, 103 at a second rate (i.e., dips per minute). Alternatively, one of disintegration-testing modules 102, 103 could stand idle while the other module 102, 103 performs operations. The independent control may be used to run multiple different test methods simultaneously on apparatus 100. The controller may record sensed dynamic information such as, for example, temperature data and invisible light detector data, at particular time intervals.

A user may interact with the controller—e.g., give commands and receive feedback—via touch screen 115. Operations specified by the controller can be for immediate execution or for time-delayed execution—as in, for example, programmed test methods. As described above, the controller receives sensor input, such as temperature data and invisible light detector data, and, in turn, implements method parameters and heater settings. The controller can also perform additional tasks such as, for example, controlling one or more indicator lights (not shown) to identify or illuminate particular disintegration-testing modules. The controller may detect the presence of modules upon connection to their corresponding connection ports in the attachment bays or during a power-up routine. The controller may receive corresponding IDs from the disintegration-testing modules via the connection ports.

Apparatus 100 may be set to automatically perform a pre-programmed procedure upon the detection of the insertion or removal of one or more disintegration-testing modules. For example, if the number of modules is changed to 1, then apparatus 100 may automatically reconfigure to operate as a single-module system. Apparatus 100 may also be set to require a technician's intervention to reprogram apparatus 100 to operate with a different number of modules. Requiring reconfiguration by a technician may be useful to prevent unauthorized modifications that may be unsafe. Requiring a technician's intervention for reconfiguration, or other actions, may be implemented by, for example, (1) requiring the entry of an authorization code on touch screen 115 or (2) the use of a hardware key (not shown) in an electro-mechanical switch (not shown) in base unit 101 that is communicatively connected to the controller to indicate engagement of the switch by the hardware key.

As noted above, a user may interact with the controller via touch screen 115. Touch screen 115 is a color touch screen, which allows for a more-varied and useful visual output to the user than a black-and-white touch screen. In addition to control of the disintegration-testing modules, the controller offers method and report storage, and multiple user-access levels to improve users' command and productivity. Touch screen 115 of FIG. 1 provides users with an interactive interface for controlling and monitoring apparatus 100. Features include storing and running methods, storing reports, managing a plurality of users at different access levels, and performing apparatus maintenance. Exemplary features of the controller, as accessed via touch screen 115, are described below. Note that, unless otherwise indicated, user input to the controller and controller output to the user is performed through touch screen 115.

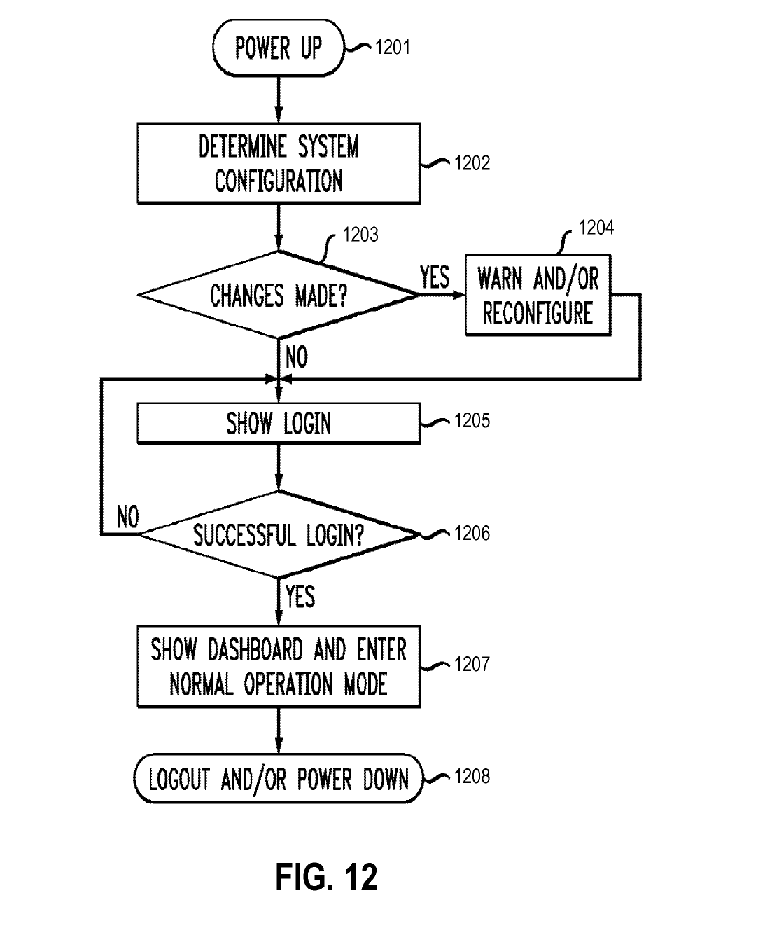

FIG. 12 shows a flowchart of an exemplary initialization operation of apparatus 100 of FIG. 1 in accordance with one embodiment of the invention. Touch screen 115—and apparatus 100 generally—may be turned on with a power button (not shown) (step 1201). Note that apparatus 100 may be powered down using the power button or by selecting a shut-down option on touch screen 115. Upon power-up, a startup check is performed by the controller to determine system configuration and status (step 1202). The controller determines which attachment bays have disintegration-testing modules—or other compatible modules—attached to base unit 101 and may obtain identification information—such as a module serial number—from each one. The controller may compare the identification information to a stored list of identifiers to determine if any changes have been made (step 1203). If changes are detected, then the controller may provide the user a warning or may offer a reconfiguration option (step 1204). Next, touch screen 115 shows a login screen for user login (step 1205). The login screen may have a text-entry box for typing in a user name or a selection box (e.g., drop-down or scrollable list) for selecting a user identifier (e.g., name). Touch screen 115 may show a corresponding access level for each user. After a user identifier is entered or selected, the user may be prompted to enter a corresponding passkey in a text-entry box or on a keypad.

If the login is successful (step 1206), then touch screen 115 shows an operations window—described below—and enters normal operation mode, which allows user interaction with apparatus 100 at a level that corresponds to the user's access level (step 1207). The operations window is a multi-tab screen that starts on the dashboard tab, described below. If login is unsuccessful (step 1206), then touch screen 115 returns to step 1205—showing the login screen. When a user is done with a session, the user may log out and/or power down apparatus 100 (step 1208). Note that if a test method is running, then the logout and shutdown options are made unavailable to the user. Unavailability of options generally may be indicated by, for example, graying out the corresponding buttons on touch screen 115, deleting them, or otherwise changing their visual appearance on touch screen 115. Note that mechanisms may be provided to allow certain users to log out and/or power down apparatus 100 even if a test method is running.

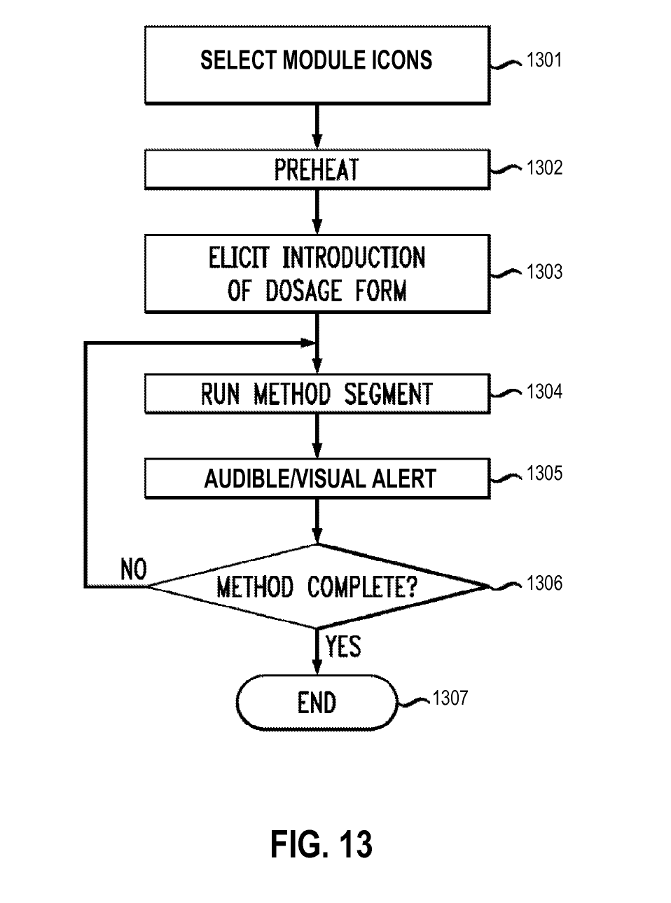

FIG. 13 shows a flowchart of an exemplary method-run operation of apparatus 100 of FIG. 1 in accordance with one embodiment of the present invention. Once a method to be run is selected, the method run starts with the selection of the icons for the disintegration-testing modules that will run the method (step 1301). After all the modules are selected, the user indicates readiness for the next phase and the method starts with a preheat phase (step 1302), in which the beaker contents are heated to a specified temperature. Once the appropriate temperature has been reached, touch screen 115 shows the corresponding module icons as flashing. In addition, user interface 114 may sound a corresponding audible alert using a speaker or other sound generator (not shown).

Depending on method parameters, the user is instructed to appropriately introduce dosage forms into the corresponding module (step 1303). The dosage forms may also be introduced automatically using an appropriate automatic dosage-form dispenser (not shown). After the dosage forms are introduced, the method run is started and a method segment is run (step 1304). When disintegration is complete, the corresponding module icons are highlighted and a corresponding audible alert may be sounded (step 1305). Icon highlighting may be indicated by, for example, flashing or otherwise visually altering the icon. If the method is completed (step 1306), then the method run terminates (step 1307); otherwise, the method run continues as above with another method segment (step 1304).

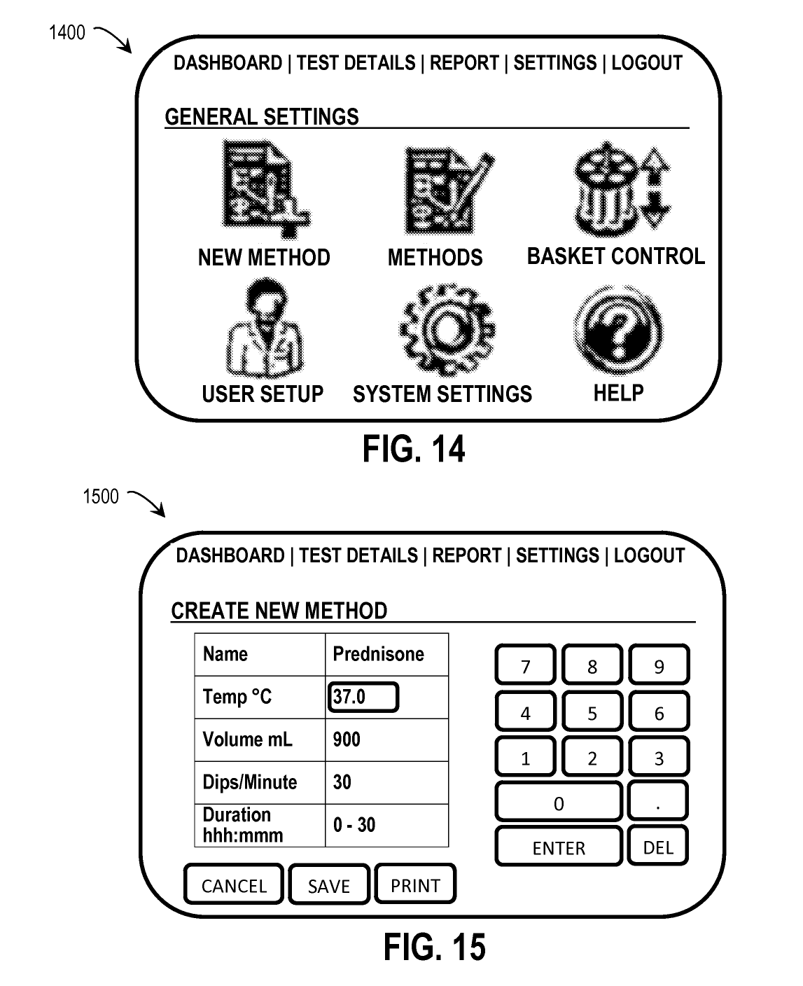

FIG. 14 shows an exemplary general settings window 1400 on touch screen 115 of FIG. 1, after selecting the settings tab. Window 1400 has a title indicating that it is a general settings window and includes selectable icons to allow a user to: (1) create a new test method, (2) edit an existing test method, (3) manually raise/lower one or more basket assemblies, (4) view and/or change user accounts, (5) view and/or change system settings, and (6) view help information.

FIG. 15 shows an exemplary new method creation window 1500 on touch screen 115 of FIG. 1. Window 1500 has entry fields and a numeric pad area for creating a new method, including drug name, temperature, volume, dips per minute, and duration. Selectable buttons are provided to cancel, save, or print.

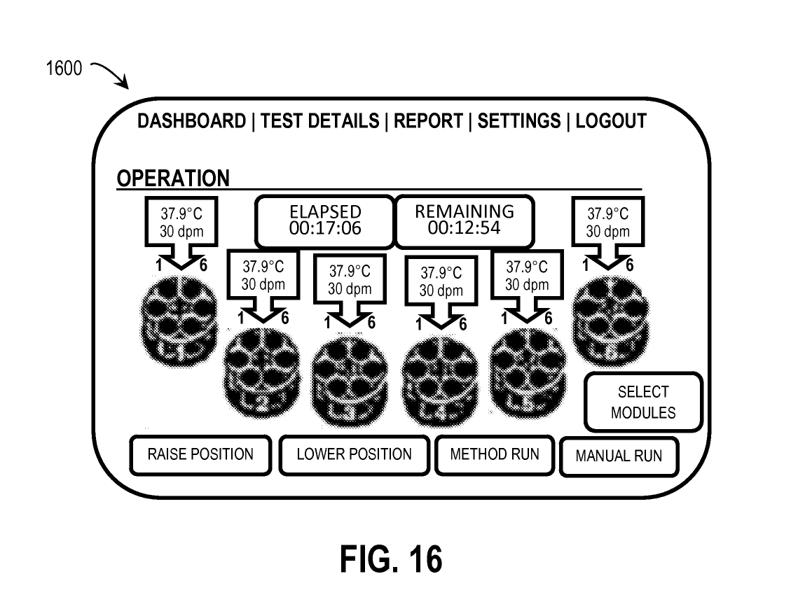

FIG. 16 shows an exemplary operations window 1600 on touch screen 115 of FIG. 1. Window 1600 shows information for apparatus 100, including which attachment bays hold a corresponding disintegration-testing module—indicated by corresponding basket assembly icons identified by integer numbers—and sensed beaker temperature and dips per minute for the connected disintegration-testing modules (it is noted that up to six bays are accommodated in this embodiment). Note that, as described above, the corresponding modules and/or basket assemblies may be labeled with conforming numbers. If a method is running on only a subset of attached disintegration-testing modules (e.g., modules 1, 2, and 4), then those modules may be indicated by highlighting (e.g., outlining in bold) or other visual indication. If subsets of disintegration-testing modules are being used differently (e.g., running different test methods), then the different subsets may be identified by using corresponding colors for the module/basket assembly icons, where one color is used for all members of one subset and different colors are used for the other subsets. Different shadings, brightness levels, or fill patterns may be used instead of, or in addition to, colors. Modules that are idle may be grayed out. Window 1600 also includes buttons such as a raise-position button for raising the basket assemblies of a selected group of disintegration-testing modules, a lower-position button for lowering the basket assemblies of a selected group of disintegration-testing modules, a method-run button for running a selected method, a manual-run button for manually controlling operation of selected disintegration-testing modules, and a select-modules button for selecting a set of module icons. Note that, if a test method is running on a group of disintegration-testing modules, then selecting the module/basket assembly icon of any disintegration-testing module in the group may select the entire group. As noted above, under certain circumstances, any number of buttons or options may be disabled but visible, where the disability may be indicated by having the button or option grayed out. For example, the raise-shafts button and lower-shafts button may be available in manual mode but not while a method is running. Also note that similar buttons may function differently in different circumstances.

Additional and/or alternative screen views, windows, and functionality may be employed in alternative embodiments.

Exemplary embodiments have been described wherein particular elements perform particular functions. However, the particular functions may be performed by any suitable element or collection of elements and are not restricted to being performed by the particular elements named in the exemplary embodiments.

In an alternative embodiment of apparatus 100 of FIG. 1, disintegration-testing module 102 does not have a motor, and motive power is provided by base unit 101. Motive power is transferred to module 102 using means such as, for example, belts, gears, shafts, and/or electromagnetic propulsion.

In some alternative embodiments of apparatus 100 of FIG. 1, disintegration-testing module 102 does not include a heating jacket. In this scenario, the glass tubes or compartments are placed in a temperature-controlled water bath. The water bath may be a single-unit water bath or multi-unit water bath. Apparatus 100 may include a single multi-unit water bath for all attachable disintegration-testing modules (e.g., a water bath that would fit the basket assemblies of six disintegration-testing modules) or apparatus 100 may include one or more smaller multi-unit baths (e.g., a water bath that would fit the basket assemblies of two disintegration-testing modules). In some of the above-described alternative embodiments, apparatus 100 includes heating and regulating elements for the one or more water baths, where the heating is regulated by the controller of base unit 101.

In some embodiments, disintegration-testing module 102 of FIG. 1 comprises one or more visible lights (e.g., LEDs) in one or more colors. The lights may be used to illuminate the contents of the glass tubes. The lights may also be used to identify particular vessels for feedback to a user using touch screen 115 of FIG. 1 in order to, for example, highlight, a particular set of modules being monitored or programmed. The identification may be accomplished by using lights of substantially the same color as the color of the corresponding vessel icons on touch screen 115.

In some alternative embodiments of apparatus 100 of FIG. 1, base unit 101 has fewer or more attachment bays than two. In some embodiments, the attachment bays are arranged in a configuration other than an arced curve. In one alternative embodiment, the attachment bays are arranged as two outward facing rows. For example, one alternative modular system has a substantially rectangular footprint and comprises four attachment bays on one side and four attachment bays on the other.

Some embodiments use one or more disintegration-testing modules that do not include an agitator apparatus or motor. These disintegration-testing modules may include a vessel with a heating jacket.

In some alternative embodiments, the controller is located inside user interface 114 of FIG. 1. In some embodiments, the controller is integrated with the user-interface controller of user interface 114. In some implementations, the controller is implemented in a distributed manner, where parts of the controller are communicatively connected together and may be located in user interface 114, base unit 101, and/or elsewhere.

In some alternative embodiments, touch screen 115 of FIG. 1 is a black and white, rather than a color, screen.

In some alternative embodiments, options on touch screen 115 of FIG. 1 that are not available at a particular operational point are not visually altered. Instead, pressing those options causes a fault alert, such as an error window popping up and/or an audible alert sounding, indicating that the selected option is not available.

In some alternative embodiments, user interface 114 of FIG. 1 comprises input devices—such as, for example, buttons, slides, and/or knobs—other than a touch screen.

In some of the above-described alternative embodiments, user interface 114 does not include any touch screen. In some of the above-described alternative embodiments, user interface 114 does not include any kind of visual-output screen.

In some alternative embodiments of apparatus 100 of FIG. 1, user interface 114 is wirelessly connectable to base unit 101. In some of the above-described alternative embodiments, user interface 114 is connectable to and detachable from base unit 101. In some alternative embodiments, user interface 114 and/or the controller may be implemented as a general-purpose computer (e.g., an iPad from Apple Inc., of Cupertino, Calif.) programmed to perform above-described interface and/or controller functions.

In some alternative embodiments of apparatus 100 of FIG. 1, the detachable modules use motion and/or heating mechanisms different from the ones described above.

In some alternative embodiments of detachable module 102, the medium in the beakers is a medium other than a solution.

In some alternative embodiments of apparatus 100 of FIG. 1, the apparatus is an integrated system of non-detachable disintegration-testing modules. In these embodiments, the motor, emitters, and detectors of each disintegration-testing module (including their respective operating parameters) remain independently controllable and, consequently, the controller remains able to simultaneously run different methods on different sets of disintegration-testing modules.

It should be noted that embodiments of the invention are not limited to disintegration-testing systems. Alternative embodiments comprise modular systems other than disintegration-testing systems. Some of these alternative embodiments are dissolution-testing or other motorized pharmaceutical systems. In dissolution testing, for example, an agitator rotates instead of being a reciprocating apparatus that reciprocates up and down as in disintegration testing.

Some other of these alternative embodiments may be non-pharmaceutical motorized, modular, and scalable scientific instrumentation systems. Some of these alternative embodiments comprise a system having a central controller, base unit, and two or more attachment bays for one or more motorized modules, where the central controller receives input from, and controls operation of, the motorized modules.

References herein to the verb “to set” and its variations in reference to values of fields do not necessarily require an active step and may include leaving a field value unchanged if its previous value is the desired value. Setting a value may nevertheless include performing an active step even if the previous or default value is the desired value.

Unless indicated otherwise, the term “determine” and its variants as used herein refer to obtaining a value through measurement and, if necessary, transformation. For example, to determine an electrical-current value, one may measure a voltage across a current-sense resistor, and then multiply the measured voltage by an appropriate value to obtain the electrical-current value. If the voltage passes through a voltage divider or other voltage-modifying components, then appropriate transformations can be made to the measured voltage to account for the voltage modifications of such components and to obtain the corresponding electrical-current value.

As used herein in reference to data transfers between entities in the same device, and unless otherwise specified, the terms “receive” and its variants can refer to receipt of the actual data, or the receipt of one or more pointers to the actual data, wherein the receiving entity can access the actual data using the one or more pointers.

Exemplary embodiments have been described wherein particular entities (a.k.a. modules) perform particular functions. However, the particular functions may be performed by any suitable entity and are not restricted to being performed by the particular entities named in the exemplary embodiments.

The present invention may be implemented as circuit-based systems, including possible implementation as a single integrated circuit (such as an ASIC or an FPGA), a multi-chip module, a single card, or a multi-card circuit pack. As would be apparent to one skilled in the art, various functions of circuit elements may also be implemented as processing steps in a software program. Such software may be employed in, for example, a digital signal processor, micro-controller, or general-purpose computer.

The present invention can be embodied in the form of methods and apparatuses for practicing those methods. The present invention can also be embodied in the form of program code embodied in tangible media, such as magnetic recording media, optical recording media, solid state memory, floppy diskettes, CD-ROMs, hard drives, or any other non-transitory machine-readable storage medium, wherein, when the program code is loaded into and executed by a machine, such as a computer, the machine becomes an apparatus for practicing the invention. The present invention can also be embodied in the form of program code, for example, stored in a non-transitory machine-readable storage medium including being loaded into and/or executed by a machine, wherein, when the program code is loaded into and executed by a machine, such as a computer, the machine becomes an apparatus for practicing the invention. When implemented on a general-purpose processor, the program code segments combine with the processor to provide a unique device that operates analogously to specific logic circuits.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless explicitly stated otherwise, each numerical value and range should be interpreted as being approximate as if the word “about” or “approximately” preceded the value of the value or range. As used in this application, unless otherwise explicitly indicated, the term “connected” is intended to cover both direct and indirect connections between elements.

For purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. The terms “directly coupled,” “directly connected,” etc., imply that the connected elements are either contiguous or connected via a conductor for the transferred energy.

The use of figure numbers and/or figure reference labels in the claims is intended to identify one or more possible embodiments of the claimed subject matter in order to facilitate the interpretation of the claims. Such use is not to be construed as limiting the scope of those claims to the embodiments shown in the corresponding figures.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

Although the steps in the following method claims are recited in a particular sequence with corresponding labeling, unless the claim recitations otherwise imply a particular sequence for implementing some or all of those steps, those steps are not necessarily intended to be limited to being implemented in that particular sequence.

1. A disintegration testing apparatus comprising:
at least one area for a compartment, the compartment adapted to hold a dosage form during disintegration testing; and
at least one light emitter and at least one light detector arranged around each of the areas so as to face the area;
wherein the at least one light detector is adapted to provide, when a compartment is disposed within the area, a signal indicative of the amount of detected light provided by the at least one light emitter.

2. The apparatus of claim 1, wherein:
the at least one light detector comprises three light detectors arranged on a first side of the area; and
the at least one light emitter is arranged on a second side of the area opposite the first side.

3. The apparatus of claim 1, wherein the at least one light emitter is an infrared light emitter, and the at least one light detector is an infrared light detector.

4. The apparatus of claim 1, further comprising a microcontroller or processor coupled to control the operations of the at least one light emitter and the at least one light detector.

5. The apparatus of claim 1, further comprising a bottom flange, wherein:
the at least one area comprises two or more areas of the bottom flange, each area for a respective compartment adapted to hold a dosage form during disintegration testing;
at least one light emitter and at least one light detector are arranged around each area;
the light emitters and light detectors are mounted to the bottom flange; and
the bottom flange is encapsulated in a material that permits the bottom flange to be immersed in a liquid medium during disintegration testing without the liquid contacting the light emitters and light detectors.

6. The apparatus of claim 4, wherein the microcontroller or processor is adapted to receive signals from the at least one light detector and to determine, based on the signals, at least one of: (i) whether a dosage form in the compartment is moving, and (ii) whether a dosage form in the compartment is blocking at least one of the light detectors.

7. The apparatus of claim 4, wherein the microcontroller or processor is adapted to:
turn off the at least one emitter;
detect ambient light using the at least one light detector;
store a reference value representing the detected light; and
subsequently, during disintegration testing, offset a detected light value by the stored reference value.

8. The apparatus of claim 4, wherein the microcontroller or processor is adapted to:
alternatingly toggle the at least one emitter on and off based on a predetermined timing;
detect ambient light during an off period of toggling using the at least one light detector;
store a first value representing the light detected during the off period;
detect light during an on period of toggling using the at least one light detector;
store a second value representing the light detected during the on period; and
store the difference between the first and second values as a third value.

9. The apparatus of claim 1, wherein the microcontroller or processor is further adapted to:
alternatingly toggle the at least one emitter on and off based on a predetermined timing pattern during at least one of (i) calibration and (ii) disintegration testing.

10-12. (canceled)

13. A bottom flange for disintegration testing equipment, the bottom flange comprising:
a substrate having at least one area for a respective compartment, the compartment adapted to hold a dosage form during disintegration testing;
at least one light emitter and at least one light detector arranged around each area and mounted to the substrate; and
a material encapsulating the at least one light emitter, the at least one light detector, and the substrate to (i) permit the bottom flange to be immersed in a liquid medium during disintegration testing without the liquid contacting any of the at least one light emitter and at least one light detector, and (ii) permit light from the at least one light emitter to pass through the material and be received by the at least one light detector.

14-15. (canceled)

16. A method for auto-detection in disintegration testing, comprising:
providing a signal from at least one light detector, the signal indicative of the amount of detected light provided by at least one light emitter;
wherein the at least one light emitter and at least one light detector are arranged around and facing an area for a compartment, the compartment adapted to hold a dosage form during disintegration testing.

17. The method of claim 16, further comprising:
receiving signals from the at least one light detector; and
determining, based on the signals, at least one of: (i) whether a dosage form in the compartment is moving, and (ii) whether a dosage form in the compartment is blocking at least one of the light detectors.

18. The method of claim 16, further comprising:
turning off the at least one emitter;
detecting ambient light using the at least one light detector;
storing a reference value representing the detected light; and
subsequently, during disintegration testing, offsetting a detected light value by the stored reference value.

19. The method of claim 16, further comprising:
alternatingly toggling the at least one emitter on and off based on a predetermined timing;
detecting ambient light during an off period of toggling using the at least one light detector;
storing a first value representing the light detected during the off period;
detecting light during an on period of toggling using the at least one light detector;
storing a second value representing the light detected during the on period;
storing the difference between the first and second values as a third value.

20. The method of claim 16, further comprising:
alternatingly toggling the at least one emitter on and off based on a predetermined timing pattern during at least one of (i) calibration and (ii) disintegration testing.

21. The apparatus of claim 8, wherein the microcontroller or processor is further adapted to compare a stored reference value to the third value to provide a present indication of the state of the dosage form.

22. The apparatus of claim 21, wherein the microcontroller or processor is further adapted to provide an indication that the dosage form has disintegrated based on a plurality of comparisons over a predetermined period of time of the stored reference value to a plurality of third values determined over the predetermined period of time.

23. The method of claim 19, further comprising comparing a stored reference value to the third value to provide a present indication of the state of the dosage form.

24. The method of claim 23, further comprising providing an indication that the dosage form has disintegrated based on a plurality of comparisons over a predetermined period of time of the stored reference value to a plurality of third values determined over the predetermined period of time.
